(19) United States
(12) Patent Application Publication
(10) Pub. No.: US 2003/0065241 A1
(43) Pub. Date: Apr. 3, 2003

(54) Medical risk assessment system and method

(76) Inventor: Joerg Hohnloser, San Francisco, CA (US)

Correspondence Address: GALLAGHER & LATHROP, A PROFESSIONAL CORPORATION, 601 CALIFORNIA ST, SUITE 1111, SAN FRANCISCO, CA, 94108, US

(21) Appl. No.: 10/220,204

(22) PCT Filed: Mar. 2, 2001
(86) PCT No.: PCT/US01/04938

(51) Int. Cl. A61N 5/00

(52) U.S. Cl. 600/1

(57) ABSTRACT

A method of assessing risk for an individual to experience a specific outcome within a disease entity within a specified time frame is provided. Peer-reviewed scientific publications are analyzed to identify pertinent risk factors (212) for developing disease processes and their possible complications. Information that characterizes an individual (218) in relation to the identified risk factors is then received, preferably responsive to question (224) regarding any demographic values of an individual under test and questions regarding medical chracterisitics of the individual under test. An estimate of risk of the individual acquiring the outcome within a specified time frame is performed based on the identified plurality of risk factors (212). Assessment of medical risk and condition may include analyzing peer-reviewed scientific publications to identify populations affected by a medical outcome and for each population respective risk factors that affect risk of acquiring the medical outcome within a specified time-frame, associating an individual with one of the identified population, identifying information that characterizes the individual (218) in relation to the respective risk factors other associated population, and estimating risk of the individual having the medical outcome within the specified the time frame responsive to the identified information. Promotion of business on a site of a computer network is provided by supplying an on-line questionnaire (224) regarding characteristics of individual (218) under test, receiving information regarding characteristics of the individual under test, and responsive to the received information, providing an assessment of the individual under test having a medical outcome within a specified time frame.

### TECHNICAL FIELD

The present invention pertains to assessment, diagnosis, recommendation, and treatment of conditions that may be specific to a disease entity.

### BACKGROUND ART

Risk assessment, diagnosis, and treatment of conditions, particularly as it pertains to conditions specific to one or more disease entities, is an area that has proven difficult to reduce to scientific or algorithmic precision. This is true for patients and physicians alike.

Risk assessment by the patient has conventionally been highly inaccurate. Most patients have no experience or knowledge of the broad cross-spectrum of outcomes that they might experience. Patients thus frequently misunderstand the importance of symptoms, or come to highly inaccurate conclusions about their current condition and the likelihood of experiencing a particular outcome in the future. Indeed, patients may not even consider the broad cross-section of potential outcomes and associated risk factors because they are unaware of their existence or do not appreciate their significance.

Risk assessment by the physician is typically better than that of the patient, but is prone to inaccuracies as well. In the typical physician—patient relationship, the physician may receive data about the patient from a variety of sources, such as patient history and complaints, physical examination, prior medical records, and test results. In response to the patient specific data, the physician may judge the patient's condition and medical needs using the medical expertise developed through medical training and the practice of medicine. The physician may use medical treatises and scientific literature to further substantiate his or her opinions.

Frequently, these judgments are significantly influenced by the physician's own experience taking care of patients with similar presentations. It is impractical for the treating physician to analyze each patient's outcome by performing a detailed literature search of pertinent medical publications and scientific data, and then to apply the results of such research to a particular patient situation. Moreover, it is impossible for any one physician to analyze even a substantial cross-section of the scientific data available in accepted medical journals due to the enormous quantity of scientific data about human disease that has been developed over the last few decades. Consequently, risk assessment is frequently handled in a crude manner. The physician's own biases frequently affect his or her risk assessments, diagnostic recommendations, and therapeutic recommendations.

Due to these and other considerations, there is frequently substantial disagreement among physicians regarding the appropriateness of using particular diagnostic tests and treatments. The under-utilization and inappropriate utilization of diagnostic tests and treatments is believed to substantially and negatively impact the health of medical consumers by failing to deliver potentially helpful data about patient diagnosis and/or response to therapy. On the other hand, the over-utilization of diagnostic tests and treatments greatly increases the costs of medical services, and exposes patients to drug and/or procedural implications that may be unnecessary.

### DISCLOSURE OF INVENTION

According to a first aspect of the present invention, a method is provided for generating an epidemiological database. According to the method, peer-reviewed scientific literature is searched to identify a class of studies that include data about a risk of experiencing an outcome specific to a disease entity for members of a demographic group. The data is extracted from the various publicly available scientific sources such as country-specific census data and WHO data.

According to a second aspect of the present invention, a method is provided for assessing a risk that an individual will experience an outcome specific to a disease entity within a specified period of time. The method uses a database of epidemiological data extracted from peer-reviewed scientific literature. According to the method, a demographic profile is identified that specifies a demographic group to which the individual belongs. A baseline risk that the individual will experience the outcome over the specified period of time is determined in response to the demographic profile. The database is searched to identify a best matching study in said literature for the demographic group. Any epidemiological data for said demographic group that was extracted from the best available study is retrieved from the database. An epidemiological profile that provides values for any individual specific variables included in the identified epidemiological data is identifed. Finally, the baseline risk is adjusted in response to the values and epidemiological data to generate the assessed risk.

According to a third aspect of the present invention, a method is provided for assessing a risk that an individual will experience an outcome specific to a disease entity within a specified period of time. According to the method, a demographic profile that specifies a demographic group to which the individual belongs is identified. A baseline risk that the individual will experience an outcome specific to a disease entity over a specified period of time is determined in response to the demographic profile. A database of risk factors extracted from peer-reviewed scientific literature is searched to identify a plurality of risk factors for that demographic group and outcome. The risk factors have been extracted from a best available study in said literature for that demographic group. An epidemiological profile is identified that provides values for the identified risk factors for the individual. Finally, the baseline risk is adjusted in response to the values to generate the assessed risk.

According to a fourth aspect of the present invention, a method of recommending a course of medical action for an individual is provided. The method uses a database of risk data that includes data about risk factors and corresponding risk factor effects, said risk data having been extracted from peer-reviewed scientific literature. According to the method, a first set of data that characterizes an individual in relation to a plurality of demographic parameters is identified. The database of epidemiological data extracted from peer-reviewed scientific literature is searched in response to the first set of data to identify a plurality of risk factors and corresponding risk factor effects that are adjusted for interdependency among the risk factors. A second set of data that characterizes the individual in relation to the plurality of risk factors is identified. In response to the sets of data, a course of medical action is recommended for the individual.

According to a fifth aspect of the present invention, a method of promoting business on a computer network is provided. The method comprises supplying a first online questionnaire to a user about demographic characteristics of an individual, receiving a response to the first questionnaire and in response thereto searching epidemiological data extracted from peer-reviewed scientific literature to identify a plurality of risk factors that affect the likelihood of the individual experiencing a specified outcome, supplying a second online questionnaire to the user about the epidemiological characteristics of the individual relative to the identified plurality of risk factors, receiving a response from the user to the second questionnaire, and assessing risk for the individual to experience a specified outcome within a disease entity within a specified period of time in response to both responses from the user.

According to a sixth aspect of the present invention, a computer is programmed to receive a demographic profile of an individual; and responsive to the profile, to supply a questionnaire about a plurality of risk factors specific to a specified disease entity; and responsive to a response to the questionnaire, to supply an assessment of the risk of the individual experiencing at least one outcome specific to the specified disease entity over at least one specified period of time.

Risk assessment according to the present invention may be implemented by any of a wide variety of supporting infrastructure, such as stand-alone computers, computer networks, Internet based embodiments, personal digital assistants (PDAs), and embedded systems. The present invention may be implemented in a portable manner to operate across a broad class of platforms, such as personal computers (PCs), Macintosh computers, computer workstations such as those supplied by SUN Microsystems, and mainframe computers. The present invention may also be carried on computer or other machine-readable media Examples of machine readable media include magnetic storage media such as floppy disks, hard disks, and magnetic tape, magneto-optical storage media such as minidisk, optical storage media such as the compact disk (CD) and digital versatile disk (DVD). Other examples of machine-readable media include magnetic, electric, and optical communication links, such as conventional twisted pair, coaxial cable, optical cable, and wireless communication channels. Such machine-readable media may carry a program of instructions executable by a machine for performing risk assessment according to the present invention. The form of the supporting structural implementation is not important to the invention.

The various features of the present invention and its preferred embodiments may be better understood by referring to the following discussion and the accompanying drawings. The contents of the following discussion and the drawings are set forth as examples only and should not be understood to represent limitations upon the scope of the present invention.

### BRIEF DESCRIPTION OF DRAWINGS

FIG. 1A a schematic diagram illustrating an embodiment of a risk assessment system.

FIG. 1B is a flowchart illustrating an embodiment of a method of assessing the risk that an individual will experience a specified outcome specific to a disease entity within one or more specified periods of time.

FIG. 2 is a schematic diagram of a risk assessment system.

FIG. 3A is a flow chart illustrating an embodiment of a method of generating an epidemiological database.

FIG. 3B is a flow chart illustrating an embodiment of a method of searching peer reviewed scientific literature.

FIG. 3C is a schematic diagram of an embodiment of a template for evaluating studies.

FIG. 3D is a schematic diagram of another embodiment of a template for evaluating studies.

FIG. 3E is a flowchart illustrating how to extract risk data.

FIG. 4 is a flowchart illustrating another embodiment of a method of assessing the risk that an individual will experience a specified outcome specific to a disease entity within one or more specified periods of time.

FIG. 5A is a flowchart illustrating an embodiment of a method of recommending a course of medical action.

FIG. 5B is a flowchart illustrating an embodiment of a method of assessing the risk that an individual will experience a specified outcome within a specified period of time, and optionally of recommending a course of medical action.

FIG. 5C is a flowchart illustrating an embodiment of a method of providing diagnostic and/or therapeutic recommendations.

FIG. 6 is a flow chart illustrating an embodiment of a method of promoting business.

### MODES FOR CARRYING OUT THE INVENTION

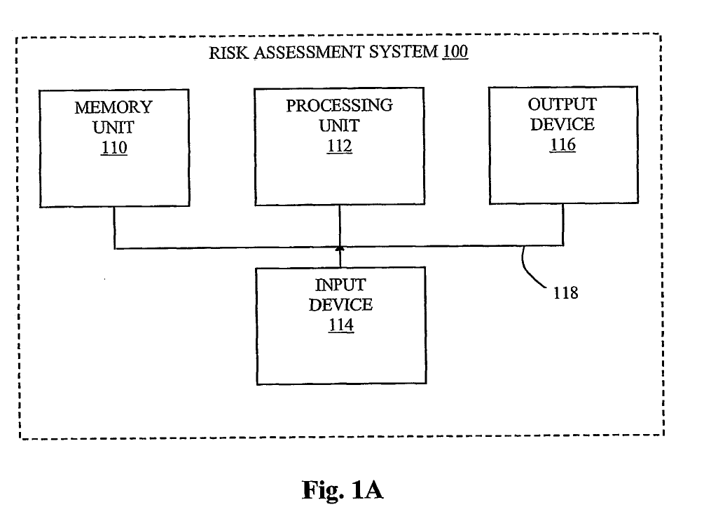

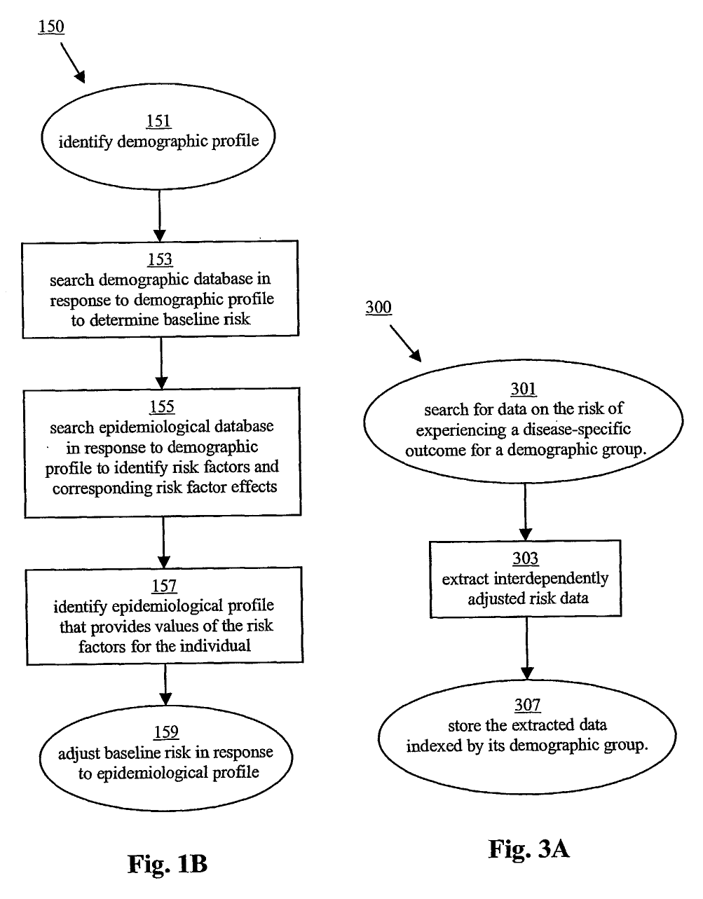

Referring now to FIG. 1A there is shown an embodiment of a risk assessment system 100. The system 100 comprises a processing unit 112, a memory unit 110, an input device 114, and an output device 116, interconnected in conventional manner by a bus 118. The input device 114 and output device 116 may be of conventional design. The processing unit 112 may be a general-purpose central processing unit, such as the Pentium® III manufactured by Intel Corporation. The memory unit 110 may be a conventional random access memory (RAM) and hard-disk drive. The memory unit 110 stores an epidemiological database, a demographic database, and a program of instructions executable by the processing unit 112 for performing risk assessment in accordance with the present invention. The databases may be implemented using any of a wide variety of commercially available database or spreadsheet programs, such as Microsoft Access® or Microsoft Excel®. The program of instructions may be implemented in any of a wide variety of programming languages, such as C, C++, or Basic. Data for the demographic database may be extracted from sources such as hospital statistics, World Health Organization (WHO) statistics, and census data. Data for the epidemiological database may be extracted from peer reviewed scientific literature. The best available evidence preferably is used to construct the databases.

The system 100 may be implemented by any of a wide variety of supporting infrastructure, such as a stand-alone computer, computer network, Internet based embodiment, personal digital assistant (PDA), and/or an embedded system. The present invention may be implemented in a portable manner to operate across a broad class of platforms, such as one or more personal computer (PC), Macintosh computer, computer workstation, and/or mainframe computer.

Referring now to FIG. 1B, there is shown a flowchart that illustrates an embodiment of a method 150 of assessing the risk that an individual will experience a specified outcome specific to a disease entity within one or more specified periods of time. The method 150 may be implemented using the risk assessment system 100 of FIG. 1A.

In method 150, a demographic profile is identified 151 that provides demographic data about the individual. The demographic profile may specify an age, gender, ethnicity, and/or geographic region of residence of the individual. A database of demographic data is searched 153 using the demographic profile as a search query. This search identifies data that determines a baseline risk that the individual will experience the outcome over one or more specified periods of time. The baseline risk may be included in the data itself, or computed from the data using Equations 2 and 3 (given below) or in another conventional manner. The epidemiological database is searched 155 using the demographic profile as a search query. This identifies a set of risk factors that affect the risk that the individual will experience the specified outcome, and identifies the corresponding risk factor effects. The risk factors preferably are those that pertain to members of the same demographic group as the individual, (which, for example, may be determined to be those persons having the same demographic profile as the individual). The corresponding risk factor effects and prevalence rates are retrieved from the epidemiological database. An epidemiological profile is identified 157 that provides values of the risk factors for the individual. The risk that the individual will experience the specified outcome is assessed by adjusting 159 the baseline risk. The adjustment may be determined from the risk factor effects, prevalence rates, and the various values provided in the questionnaires. Equations 4 through 11 (given below) may be used to determine the adjusted risk.

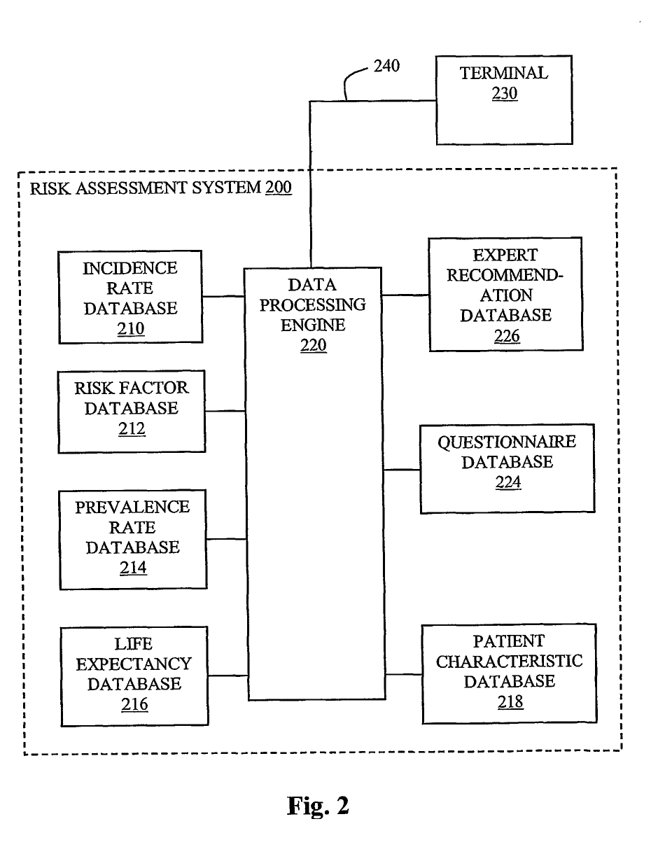

Referring now to FIG. 2, there is shown a schematic diagram that illustrates another embodiment of a risk assessment system 200. The system 200 may be implemented on virtually any general-purpose computer system, computer network, or personal digital assistant (PDA). The system 200 comprises a demographic database and an epidemiological database. The demographic database comprises an incidence rate database 210 and a life expectancy database 216. The epidemiological database comprises a risk factor database 212 and a prevalence rate database 214. The risk factor database stores sets of risk factors and corresponding risk factor effects indexed according to the demographic group(s) to which they pertain. A patient characteristic database 218, a questionnaire database 224, and an expert recommendation database 226 are also included in this embodiment of the system 200. The system 200 also comprises a data processing engine 220 which performs various data processing operations in conventional manner, such as the execution of instructions, searching of databases, numerical and logic computations, and the receipt, storage, retrieval, transmission, and/or other processing of information. These various databases are interconnected with the data processing engine 220 in a conventional manner. The data processing engine 220 is connected via a communications link 240 with a terminal 230. While one terminal 230 is shown, those having ordinary skill in the art of electronics will appreciate that any number of terminals could be linked to the data processing engine 220 using any of a wide variety of communications links. While a plurality of databases are shown, they may be stored in a single database file, such as a large spreadsheet. The number of files used is not material to the present invention.

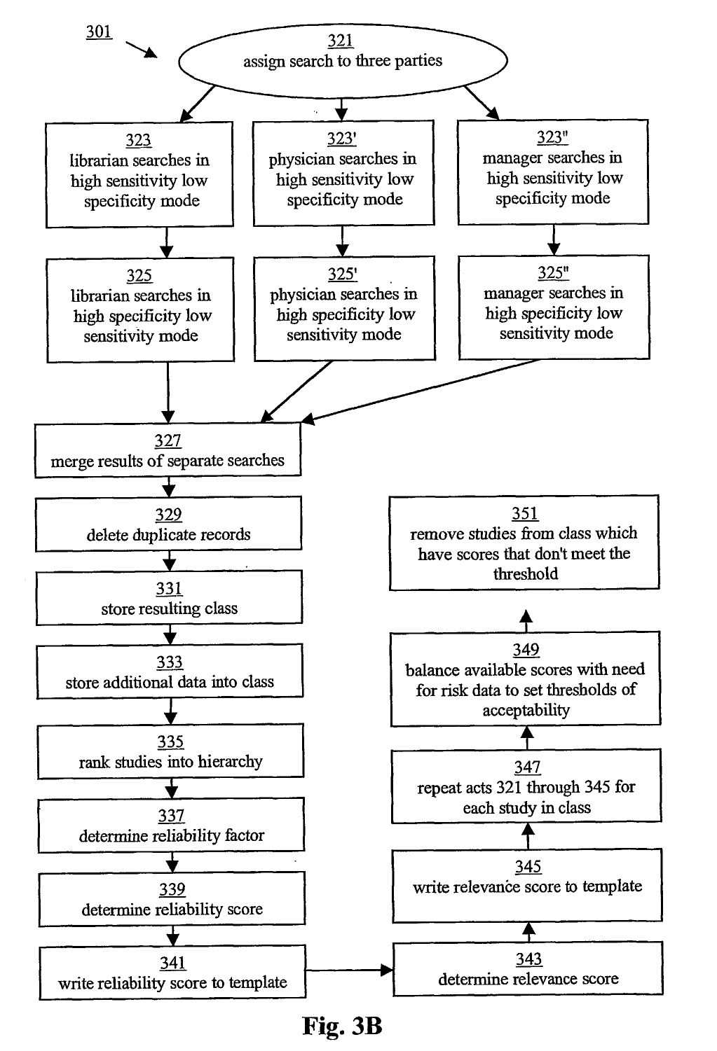

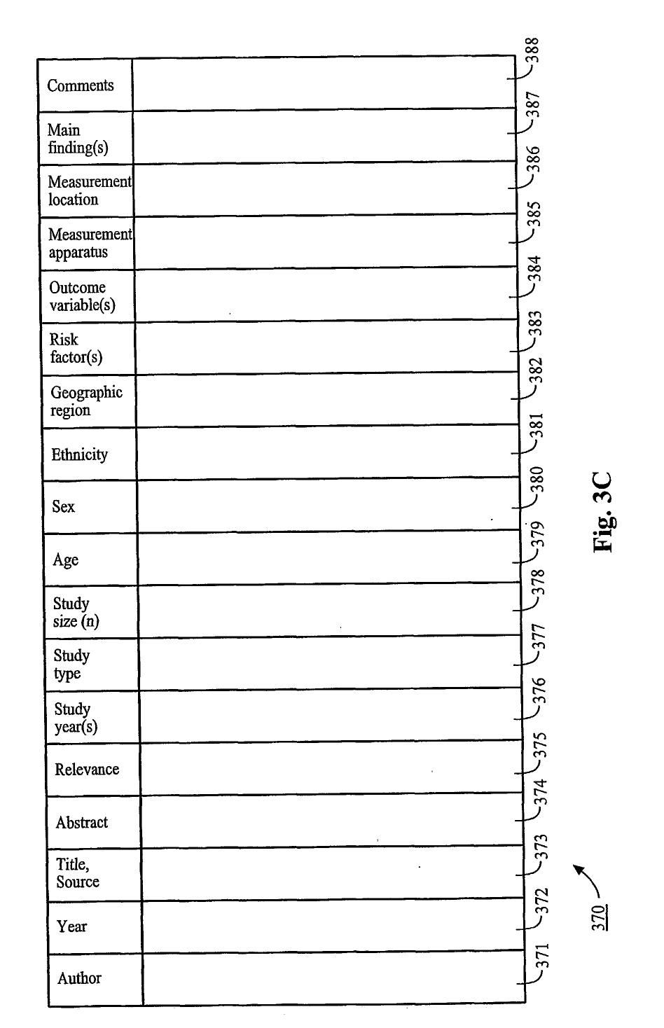

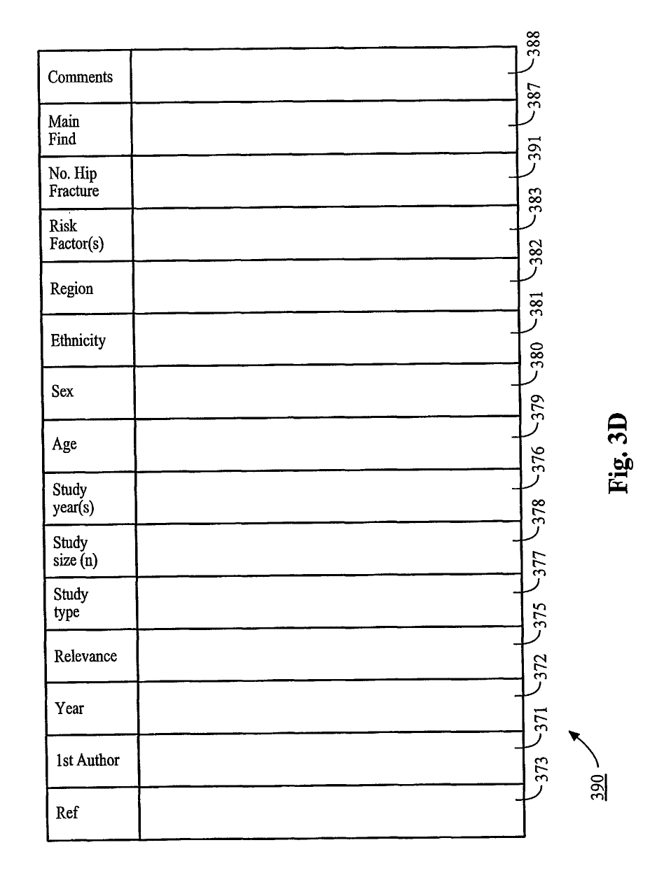

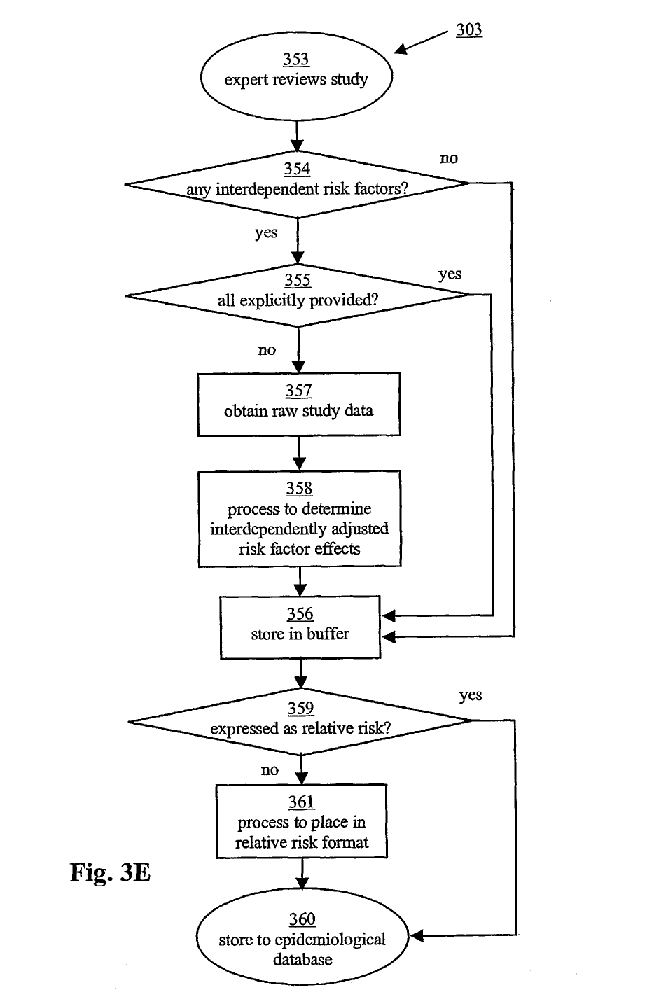

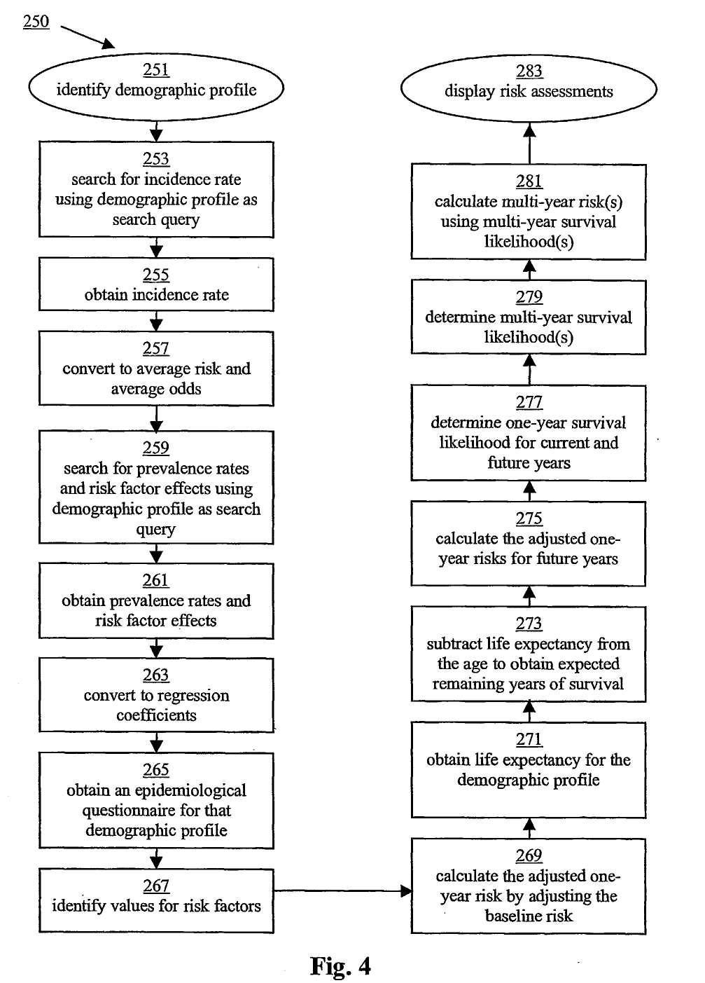

Preparation of data for the various databases 210, 212, 214, 216, 218, 224, 226 is discussed next with respect to FIGS. 3A, 3B, 3C, 3D and 3E. Various modes of operation of the system 200 are discussed with respect to FIGS. 4, 5A, 5B, 5C and 6.

Referring now to FIG. 3A, there is shown a flowchart that illustrates an embodiment of a method 300 of generating data for the risk factor database 212, prevalence rate database 214, and questionnaire database 224. A collection of peer-reviewed scientific literature is searched 301. The search identifies a class of studies that include data about the risk factors for experiencing outcomes specific to the specified disease entity. The data also identifies the demographic group to which the risk factors pertain. The data preferably also includes the risk factor effect and the prevalence rate that correspond to each risk factor. A risk factor effect indicates an effect that the corresponding risk factor has on the risk of experiencing the outcome for members of the demographic group. The effect may be dependent on whether (or the extent to which) a member exhibits the risk factor. The effect may be relative to members of the same demographic group that do not exhibit the risk factor. The prevalence rate indicates the mean (or other average) rate or probability that members of the demographic group exhibit the risk factor.

The studies in the class are evaluated to determine their reliability. Each study having reliability beneath a specified threshold is removed from the class. The data is extracted 303 from the studies in the class. The extracted data is in a form that accounts for any interdependencies among the risk factors. For example, the values of the risk factor effects may be adjusted to account for such interdependencies. Data may be extracted for each demographic group analyzed in the study. The data is stored 307 in the epidemiological database indexed by the demographic group to which the data pertains. Questions and answers for determining the values of risk factors for individuals under test may also be extracted from the studies. The questions and answers preferably are as close as is practical to those used in the studies themselves. The question and answers are indexed by demographic group and stored in the questionnaire database 224.

The stored data may be used for a variety of functions, such as risk assessment, risk enrichment, and the recommendation of diagnostic evaluations and therapies suitable to the risk profile and medical history of an individual. For example, the risk that a given individual will experience a disease specific outcome may be assessed as follows. First, the individual's demographic group is identified. A user of the system 200 is then asked a set of questions specified in a study that analyzed that demographic group. Preferably, the questions come from the best matching study for that demographic group. A set of possible answers for each question is supplied with the questions. The answers preferably are extracted from the same study as the questions. The user selects the answers that best match the given individual's condition. The selected answers are processed by the system 200 to assess the risk that this individual will experience the outcome over one or more periods of time.

Referring now also to FIG. 3B, there is shown a flowchart that illustrates an embodiment of the act 301 of searching a collection of peer-reviewed scientific literature. The development of a search query is assigned 321 to a medical librarian, a physician, and a search team manager. Each of these parties independently develops a search query to identify the best available scientific data about a specified disease and its specific outcomes.

The search queries are used to search a collection of peer reviewed scientific literature. The collection may be the holdings of a university library or a professional medical database such as MEDLINE, for example. University libraries and professional medical databases have established effective search infrastructures to assist with the search. It is considered acceptable to search the study abstracts to identify relevant studies provided that abstract data is not used in risk assessment computations. A first stage 323, 323′, 323″ of the search operates in a “high sensitivity and low specificity” mode. This captures the substantial majority of those studies in the collection that are related to the specified disease. The volume of captured material is reduced in a second stage 325, 325′, 325″ of the search. The second stage 325, 325′, 325″ may operate in a “high specificity and low sensitivity” mode. Multiple specific searches may be run to capture a smaller and more relevant percentage of the collection.

The results of the searches are merged 327 to form an initial specification of the contents of a relevant class of studies. Duplicate results are deleted 329 from the initial specification. The identity of each study in the resulting class of studies is stored 331 in a search database. This database does not need to be included in the risk assessment system 200. Virtually any commercially available database or spreadsheet program may be used to implement this database. The studies in the initial specification are acquired for evaluation.

Further searching may be conducted to find relevant but missed references. These may be added to the class. Preferably, the results of such search are supplemented by data from a variety of other reliable sources of medical data, such as the HEALTHSTAR medical database and the COCHRANE LIBRARY. MEDLINE, HEALTHSTAR, and the COCHRANE LIBRARY are well known and frequently used by physicians and others having ordinary skill in the medical and research arts. Additional reliable data may be obtained from other sources, such as the opinions of acknowledged experts in a field of medicine, and added 333 to the class and stored in the search database.

Next, the studies in the class are evaluated for reliability and relevance. Referring now also to FIG. 3C, there is shown a schematic diagram of an embodiment of a template 370 that is useful for formally evaluating the studies. The template 370 is particularly well suited to studies that provide data about the Bone Mineral Density of study participants.

A set of fields 371 through 388 is provided in the template 370. Each of the fields has a corresponding heading. Fields 371, 372, and 373 are provided for the author or authors of the study, the year of publication, and the title and source of the study. Field 374 may be used either to indicate whether the study has an abstract, or alternatively, to rank the relevance of the study based on the content of its abstract. A study may address more than one demographic group over more than one specified period of time. Each demographic group that has been separately analyzed in the study is identified. Study data for distinct demographic groups is written in distinct vertical regions of the template 370. Lines may be added to the template 370 to separate the vertical regions. The age or age range, gender, ethnicity, and/or geographic region of residence of residence of participants in the demographic group is written to the corresponding field 379, 380, 381, and 382 respectively. The years during which the demographic group was studied are written to field 376, and the number of participants in the group is written to field 378.

While the template 370 is primarily valuable during the evaluation process, room is provided in field 387 under the heading “Main finding(s)” for risk data provided in the study. A pointer, such as “see Table 1.2” may be alternatively be provided under this heading where, for example, the extracted risk data is too voluminous to fit in the space provided in field 387. Examples of such voluminous results are large tables of T-Scores (T-scores) and/or large tables of Odds Ratios (OR) which may be too large to fit in the space provided on the template 370. The template 370 may be implemented in any of a wide variety of manners, including printed forms and computerized forms. A link may be provided so that the corresponding data may be accessed quickly.

Referring again to FIG. 3B, studies in the class are evaluated to determine their reliability. Each study considered not to be sufficiently reliable is removed from the class. This may be implemented by removing each study whose reliability is beneath a specified threshold. Determination of the-reliability of a study is based substantially on the skills of trained medical experts, such as medical physicians and licensed therapists. It is believed to be preferable to rank 335 the studies in the class. For example, the following hierarchy may be used. Studies which document meta-analysis of randomized controlled trails are often the most reliable and are assigned the rank of one. Studies which document a single randomized controlled trial are often the next most reliable and accordingly are assigned the rank of two. Cohort studies are assigned the rank of three. Case controlled studies are assigned the rank of four. Other studies or evidence, such as expert opinions not documented according to peer reviewed protocols, are assigned the rank of five or more. The study type and rank is written to field 377 of the template 370.

The hierarchy may be dependent on the particular disease, outcomes, and/or risk factors being addressed. For example, some of the significant risk factors that pertain to osteoporosis are the bone mineral density of the patient and the presence of hip fractures and/or vertebral fractures in the patient's medical history. For these risk factors, it is believed that the best available evidence will come from cohort studies and case controlled studies. So, cohort studies and case controlled studies are moved to the top of the hierarchy and assigned the rank one. Meta-analysis of randomized clinical trials generally does not apply for these risk factors, and is assigned the rank of five or more.

A study may be removed from the class based on specified reliability criteria. COCHRANE criteria are commonly used by trained medical professionals to evaluate the reliability of studies, and may serve as the specified reliability criteria. The criteria may be specific to the study's type. For example, cohort and case control studies with a study-size of less than one hundred participants may be neglected as not adequately representing any particular demographic group. The size of the threshold may depend on the extent that participant selection was performed in a randomized manner.

A reliability factor is determined 337 by a trained medical expert for each study group based on the reliability criteria. In this embodiment of the present invention, the reliability factor is smaller for more reliable studies, and larger for less reliable studies. The following formula may be applied to the data in the template 370 to determine 339 a reliability score given in Equation 1 as:

reliability score=rank×reliability factor (Eq. 1)

The reliability score may be modified by the trained medical expert where it appears to be inappropriate to particular features of the study or study group. The reliability score is written 341 to field 388 of the template 370.

The following reliability criteria may be used for diagnostic studies. Studies are preferred which provide a range of data relative to a widely accepted medical diagnostic test. For example, a Bone Mineral Density test is a widely accepted diagnostic test for determining whether an individual has osteoporosis. The Bone Mineral Density test is a special form of X-ray that yields a measurement of the current density of various minerals in an individual's bones. Low Bone Mineral Density is a common outcome that is specific to osteoporosis. Low Bone Mineral Density is also a risk factor for other outcomes specific to osteoporosis, such as hip fracture and vertebral fracture. Diagnostic studies which provide data on risk factors for developing low Bone Mineral Density are preferred in construction of the database. Diagnostic studies which provide a range of data on the validity of low Bone Mineral Density as an indicator of one or more outcomes specific to osteoporosis are also preferred. It is preferable if this type of discrete data is provided for participants in a study group regardless of their respective results on the diagnostic test. It is preferred if the diagnostic test has been used on an appropriate spectrum of patients, such as a spectrum of patients for whom the diagnostic test would likely be used in medical practice. Based on the reliability criteria, a reliability factor is determined 337, and applied to Equation 1 to determine 339 a reliability score. The score is written 341 in field 388 of the template 370.

The following reliability criteria may be used for prognostic studies. Studies in which a representative sample of participant patients was assembled at a common and preferably early point in the course of their disease are preferred. Studies that are thorough and that followed the patients over a period of years are preferred. The preferred length of follow-up typically depends on the particular disease and outcomes under consideration. It is also preferred if objective outcome criteria were applied in a blind manner. Objective outcome criteria, such as physical measures of deformation, help prevent tainting of study data caused by the principal investigator's emotional response to the particular deformity studied or measured. Finally, studies for which there was a validation study of an independent group of “test-set” patients are preferred. Based on these criteria, a reliability factor is determined 337 by the trained medical expert, and this reliability factor applied to Equation 1 to determine 339 a reliability score which is written 341 to field 388 of the template 370.

The studies are also evaluated for their relevance to the specific question posed by a clinical investigator. The following relevance criteria are preferred. Studies which provide risk data pertaining to an identified demographic group are preferred. It is valuable if there is solid evidence that the study group may be treated as being representative of a particular demographic group. The study methodology may be considered to determine this. It is preferred that the study provides risk data in a manner that is amenable to the generation of multivariable risk data. It is also preferable if data is provided that allows any interdependencies between risk factors to be identified.

A relevance score is determined 343 for each study question and written 345 to field 375 of the template 370. Each study of the class is evaluated 347 for reliability and relevance in the described manner. The range of the determined reliability and relevance scores may be used to identify a reliability threshold and a relevance threshold by balancing 349 the available scores with the need for risk data to include in the epidemiological database. The thresholds are allowed to be low where there is little reliable or relevant data available. However, if a vast volume of data is available for a given disease, outcome, or risk factor, then the threshold may be set high. Studies whose scores are below either respective threshold are removed 351 from the class. In Equation 1, a low score value is used to represent high reliability or relevance, and accordingly, a score is below a threshold if its numerical value is higher than the threshold.

The evaluation assures that the best available evidence is used to construct the databases. For example, for osteoporosis and its related risk data, it is believed that about ten percent of the studies identified in the initial filter based search of peer reviewed literature will prove to be both sufficiently reliable and relevant to the generation of the epidemiological database. Where little data is available for a particular disease, outcome, risk factor, or demographic group, the evaluation nonetheless identifies the best data that is available. As additional data becomes available, it may be evaluated, and the stored data may be updated.

A wide variety of alternative template formats are possible. Referring now to FIG. 3D, there is shown a schematic diagram of an alternative embodiment of a template 390. The template 390 is particularly useful for studies that address hip fracture risks specific to osteoporosis. The template 390 includes a field 391 with the heading “No. Hip Fracture”. The field 391 is used for indicating the number of hip fractures that occurred in a study group, or alternatively, the percentage of hip fractures that occurred among members of a study group. The template 390 also includes the fields 371-373, 375-383, and 387-388 which may be used for the same respective functions as in the template 370 illustrated in FIG. 3C.

Referring now also to FIG. 3E, there is shown a flowchart that illustrates an embodiment the act 303 of extracting data from the studies of a class. The text, tables, and figures, of each study in the class are reviewed 353 by trained medical and statistical experts to identify sets of risk factors, risk factor effects, and prevalence rates. For each set, the experts determine 354 whether any of the risk factors are interdependent. If the risk factors are interdependent, the experts determine 355 whether all of the corresponding risk factor effects are explicitly provided in the study in a form that accounts for interdependencies between the risk factors. If all are provided, they are stored 356 in a storage buffer together with the corresponding risk factors and prevalence rates. If some are missing, the raw data upon which the study is based is obtained 357. This data is sometimes provided in the study. Otherwise, the study authors may, in some instances, be contacted to obtain the raw data. The experts process 358 the raw data to determine a complete set of interdependently adjusted risk factor effects. Conventional statistical analysis of the raw data is used for this purpose. The interdependently adjusted risk factor effects determined in act 358 are then stored 356 to the storage buffer together with the corresponding risk factors and prevalence rates. In act 354, if all of the risk factors are indicated to be independent, they are stored 356 in the storage buffer together with their corresponding risk factor effects and prevalence rates. If there is only one identified risk factor, it is treated as being independent. The calculations performed in the study may be repeated to assure that the computational methodology used in the study is fully understood.

If interdependencies are present in the study results, but the study does not provided interdependently adjusted data, and the raw data cannot be obtained, then generating interdependently adjusted risk data is much more difficult. It is believed to be preferable not to use the study in this instance. Alternatively, adjustments may be based on data from another source, or the study data may be used without any adjustment for interdependencies.

Calculations may be used to identify prevalence of the risk factors. In study cohorts that are similar to the target population, (i.e. to the population on which the database will likely be applied), the determination of the prevalence values is straightforward. They are the same as in the study cohort. In case-control studies, prevalence rates of risk factors should be calculated from the control group, not from the cases. If a target population is dissimilar to the study population, the prevalence rates of risk factors should be obtained for the target population. This may require data external to the study.

The risk factor effects and prevalence rates may be expressed in a variety of formats. However, most studies express risk data as relative risks, and so relative risks are the storage form used for the epidemiological data in this embodiment of the present invention. To achieve this, the data stored 356 in the storage buffer is analyzed to determine 359 whether the risk factor effects and prevalence rates are expressed as relative risks. Risk factor effects and prevalence rates that are expressed as relative risks are stored 360 to the appropriate portions of the epidemiological database. Risk factor effects and prevalence rates that are not expressed as relative risks are processed 361 to place them in that format, and then stored 360 to the appropriate portions of the epidemiological database. In either case, the stored data is indexed according to the demographic group to which it pertains.

Conventional statistical formulas and techniques may be applied to processes raw data, account for interdependencies, and assess risks. Regression techniques may be used to process the multiple relative risks for each individual risk assessment procedure. A variety of regression types may be used, including linear regression, multiple regression, and logistic regression. A logistic regression formula is frequently useful for many forms of risk assessment. Linear regression formulas are often useful for Bone Mineral Density prediction. The preferred regression technique may depend on the type or types of statistical data available. The type of statistical data generally depends on the overall method in which the study was constructed. For example, where Bone Mineral Density is addressed in Osteoporosis studies, the standard deviation of the Bone Mineral Density measurement among the study population and among young adults is frequently useful.

Data for appropriately wording the questions to be included in the epidemiological questionnaires is also extracted from the studies. The wording of questions for the epidemiological questionnaires preferably is as close to the original questions used in the studies. Each question typically includes a number of possible answer foils. For example, the questions may be “multiple choice” or “true or false” questions. The number and wording of the answer foils in the questionnaires preferably is also as close as possible to that used in the study from which the corresponding questions were extracted or otherwise derived.

The questions are reviewed for usage of medical or scientific terms that likely would not be understood by the general public. In some instances, an additional set of questions is formulated that simplifies medical or scientific terms so that they will be understood by the general-public. Where it appears that such formulation might yield interview bias, it is omitted, and the subject study is tagged for use only by those who are medically or scientifically trained. The content of questions that the user sees may depend on the user's level of medical or scientific training or expertise. Each question is stored in the questionnaire database 224. The questions are indexed according to the demographic group(s) and risk factor(s) to which they pertain.

The studies are analyzed for data pertaining to diagnostic tests and therapies for the specified disease and its associated outcomes. The raw data and any statistics provided in the studies may be used to determine the reliability of the study. The diagnostic and therapeutic data that is reliable is extracted. Additional data may be obtained from common medical databases. The data is processed by a physician or other trained medical expert to generate corresponding sets of diagnostic questions and therapeutic questions.

The diagnostic questions request data about any tests that have already been conducted on the individual. They also include questions to determine whether there are diagnostic tests that cannot be performed. For example, an individual may refuse the option of taking a Bone Mineral Density test to avoid exposure to X-ray radiation. Similarly, a physician may have preferences about certain diagnostic tests, and may accordingly wish to exclude other tests from further consideration. Other considerations are the monetary cost or presence of insurance coverage for a test. The choice of which tests are to be excluded may be left to the user.

The therapeutic questions request data about particular therapies that the individual has used or is using. Additional questions may pertain to the amount and type of exercise that the individual engages in or the individual's diet. These questions may also include requests for data about contra-indications to possible therapies.

A diagnostic recommendation may be linked to each possible set of answers to the diagnostic questions, for example, by using a linked matrix of recommendations, or alternatively using a software algorithm that selects from among a plurality of diagnostic tests based on answers to question sets. A therapeutic recommendation is linked to each possible set of answers to the therapeutic questions in like manner.

The various diagnostic questions, therapeutic questions, and corresponding diagnostic and therapeutic recommendations are stored in the expert recommendation database 226. The stored data may be flagged to indicate its source. This allows the user to quickly access additional data Referring now to FIG. 4, there is shown a flowchart that illustrates another embodiment of a method 250 of assessing the risk that an individual will experience a specified outcome specific to a specified disease entity within one or more specified periods of time. The method 250 may be implemented using an embodiment of the risk assessment system 200 of FIG. 2 in which the various databases are implemented using Microsoft Excel®. Portions of the Microsoft Excel® spreadsheets are included below as tables. An example is given that addresses the disease of osteoporosis and the specified condition of hip fracture.

In method 250, a demographic profile is identified 251 that provides demographic data about the individual. The demographic profile may specify an age, gender, ethnicity, and/or geographic region of residence of the individual. The incidence rate database 210 is searched 253 using the demographic profile as a search query. As an example, consider a Caucasian male patient born in 1950 who now resides in the United States. Table 1 shows a sample of the incidence rate database 224 for different risk factor sets and different age groups. The incidence rate is given as the expected number of persons affected per 100,000. In Table 1, the ethnicity, gender, and region of residence determine the risk factor super-set (RFS). Particular data for each age group is obtained in a corresponding column of the Table 1.

TABLE 1
Portion of Incidence Rate Database 224.

| I | J | K | L | M | N | O | P | Q | R | S | T | U |
|---|---|---|---|---|---|---|---|---|---|---|---|---|
| RISK FACTOR SET | 40-44* | 45-49 | 50-54 | 55-59 | 60-64 | 65-69 | 70-74 | 75-79 | 80-84 | 85-89 | 90-94 | 95+ |
| Men II: Health Professionals | 18 | 15 | 15 | 17 | 17 | 93 | 194 | 402 | 786 | 1374 | 2080 | 2080 |
| Men I: MEDOS | 18 | 15 | 15 | 17 | 17 | 93 | 194 | 402 | 786 | 1374 | 2080 | 2080 |
| Women III: NURSES: | 5 | 10 | 26 | 52 | 66 | 218 | 409 | 796 | 1505 | 2501 | 3179 | 3179 |
| Health Study | | | | | | | | | | | | |
| Women IIa: SOF (excl. BMD) | 5 | 10 | 26 | 52 | 66 | 218 | 409 | 796 | 1505 | 2501 | 3179 | 3179 |
| or IIb_BMD (incl. BMD) | | | | | | | | | | | | |
| Men I: MEDOS | 13.0 | 10.8 | 10.8 | 12.2 | 12.2 | 79 | 186 | 368 | 491 | 318 | 965 | 965 |
| Black Women I: Northeast | 2.1 | 4.1 | 10.7 | 21.3 | 27.1 | 127 | 243 | 296 | 417 | 1231 | 859 | 859 |
| Men I: MEDOS | 13.9 | 11.6 | 11.6 | 13.1 | 13.1 | 120 | 120 | 440 | 440 | 1700 | 1700 | 1700 |
| Women I: MEDOS | 3.7 | 7.3 | 19.0 | 38.0 | 48.2 | 190 | 190 | 990 | 990 | 2410 | 2410 | 2410 |
| Men II: Health Professionals | 7.6 | 6.3 | 6.3 | 7.1 | 7.1 | 50 | 50 | 250 | 250 | 950 | 950 | 950 |
| Men I: MEDOS | 7.6 | 6.3 | 6.3 | 7.1 | 7.1 | 50 | 50 | 250 | 250 | 950 | 950 | 950 |

The specified patient is matched to the Men I: MEDOS study, which is the best matching study for Caucasian males in the United States. The search identifies the data that determines the baseline risk that the patient will experience a hip fracture in the next year. In particular, the average incidence rate (PARate) for hip fracture is obtained 255 from column L of the second row of Table 1. A Caucasian male patient born in 1950 and residing in the United States has a value of PARate_{RFS,age}=15 in a population of 100,000. The average risk (PARisk) and the average odds (PAOdds) of the occurrence of a hip fracture in the next year are determined 257 by computing Equations 2 and 3.

$$\begin{matrix}P A R i s {k}_{R F S , a g e} = \frac{P A R a t {e}_{R F S , a g e}}{100 , 000} & ( Eq . 2 ) \\ P A O d d {s}_{R F S , a g e} = \frac{P A R i s {k}_{R F S , a g e}}{1 - P A R i s k} & ( E q . 3 )\end{matrix}$$

Here, RFS is the risk factor super-set that matches the patient's country, ethnicity and gender, and the age matches the patients actual age rounded to 1 year. The computations yield PARisk=0.00015 and PAOdds=0.0001500225.

Table 2 shows the relevant portion of the risk factor and prevalence rate databases 212, 214 for a single risk factor. Table 2 is searched 259 using the demographic profile as a search query. The prevalence rate is identified in column AD, and the risk factor effect is identified in column AG. The risk factor effect and prevalence rate are obtained 261 from the epidemiological database. This process is performed on tables of the same form for the each of the other risk factors to obtain the complete set of risk factor effects and prevalence rates.

TABLE 2
Portions of Prevalence Rate and Risk Factor Databases

| AD | AE | AF | AG | AH | AI | AJ | AK | AL | AM | AN | AO | AP |
|---|---|---|---|---|---|---|---|---|---|---|---|---|
| Prevalence | 40-44 | 45-49 | 50-54 | 55-59 | 60-64 | 65-69 | 70-74 | 75-79 | 80-84 | 85-89 | 90-94 | 95+ |
| 0.500 | x | x | 0.74 | 0.74 | 0.74 | 0.74 | 0.74 | 0.74 | 0.74 | 0.74 | 0.74 | 0.74 |
| 0.580 | x | x | 0.68 | 0.68 | 0.68 | 0.68 | 0.68 | 0.68 | 0.68 | 0.68 | 0.68 | 0.68 |
| 0.850 | x | x | 0.82 | 0.82 | 0.82 | 0.82 | 0.82 | 0.82 | 0.82 | 0.82 | 0.82 | 0.82 |
| 0.650 | x | x | 0.72 | 0.72 | 0.72 | 0.72 | 0.72 | 0.72 | 0.72 | 0.72 | 0.72 | 0.72 |
| 0.960 | x | x | 0.49 | 0.49 | 0.49 | 0.49 | 0.49 | 0.49 | 0.49 | 0.49 | 0.49 | 0.49 |
| 0.800 | x | x | 0.91 | 0.91 | 0.91 | 0.91 | 0.91 | 0.91 | 0.91 | 0.91 | 0.91 | 0.91 |
| 0.810 | x | x | 0.73 | 0.73 | 0.73 | 0.73 | 0.73 | 0.73 | 0.73 | 0.73 | 0.73 | 0.73 |
| 0.910 | x | x | 0.75 | 0.75 | 0.75 | 0.75 | 0.75 | 0.75 | 0.75 | 0.75 | 0.75 | 0.75 |
| 0.570 | x | x | 0.72 | 0.72 | 0.72 | 0.72 | 0.72 | 0.72 | 0.72 | 0.72 | 0.72 | 0.72 |

The risk factors are those that pertain to Caucasian males born in 1950 and residing in the United States. The retrieved risk factor effects are expressed as relative risks RR_{i,age}, where i ranges from 1 to N, and N is the total number of relevant risk factors in this example. The relative risks are converted 263 to logistic regression coefficients using Equation 4:

b_{i,age}=ln (RR_{i,age}), (Eq. 4)

where ln is the conventional natural logarithm, i is an index representing the risk factor (or question) being, considered, RR_{i,age} is the relative risk for a specific risk factor at a specified age (such as the age of the patient), and b_{i,age} is the regression coefficient for the specific risk factor and specified age.

An epidemiological questionnaire is obtained 265 from the questionnaire database 224 and sent to the user. The user supplies answers to the various questions in the questionnaire to determine the value Xⱼ of each risk factor for the patient. In this example, the values Xⱼ are equal to integers. A range of integer values, for example, may be used to express the extent that the patent exhibits the risk factor. The system 200 uses the user's answers to identify 267 each of these values. It may take more than one question in some instances to determine the value of a given risk factor. These identified values form the epidemiological profile of the individual.

The one-year risk 1YRisk_{age} that the patient will experience a hip fracture is calculated by adjusting 269 the baseline risk as follows:

$$\begin{matrix}1 Y R i s {k}_{a g e} = \frac{1}{1 + {e}^{- c}} , \\ w h e r e & ( Eq . 5 ) \\ c = ( P O d d s + {\sum }_{j = 1}^{m} ( {b}_{j , a g e} ( {X}_{j} - {P}_{a g e} ) ) ) , & ( E q . 6 )\end{matrix}$$

and where m is the total number of risk factors for a specific risk factor set, j is an index representing the risk factor under consideration, age is the age of the patient under consideration, P_{age} is the risk factor prevalence for the patient's age, and e is the conventional exponential term.

The risk xYRisk that the individual will experience hip fracture in the next x years, is calculated using the annual risk performed for each the x of years. The annual risk uses the values of PARate_{RFS,age} wherein the age is incremented by one in each successive year. To calculate xYRisk the following operations apply. The life expectancy (LE) of the patient under consideration is obtained from the life expectancy database 216. The patient who is Caucasian, male, from the USA, and born in 1950 is matched 271 to the life expectancy LE=23.30 as shown in column U of Table 3.

TABLE 3
Portion of Life Expectancy Database 216

| H | I | J | K | L | M | N | O | P | Q | R | S | T | U |
|---|---|---|---|---|---|---|---|---|---|---|---|---|---|
| | AGE GROUP | | | | | | | | | | | | |
| IR_ethnicity | 0 | 1 | 2-4 | 5-9 | 10-14 | 15-19 | 20-24 | 25-29 | 30-34 | 35-39 | 40-44 | 45-49 | 50-54 |
| Caucasian | 73.80 | 73.3 | 69.40 | 64.5 | 59.60 | 54.8 | 50.20 | 45.5 | 40.90 | 36.3 | 31.80 | 27.5 | 23.30 |
| Caucasian | 79.60 | 79.1 | 75.10 | 70.2 | 65.30 | 60.4 | 55.50 | 50.7 | 45.80 | 41.0 | 36.30 | 31.7 | 27.30 |
| African | 66.10 | | 66.20 | 62.4 | 57.50 | 52.6 | 48.00 | 43.7 | 39.40 | 35.1 | 31.00 | 27.1 | 23.40 |
| African | 74.20 | | 74.20 | 70.3 | 65.40 | 60.5 | 55.70 | 50.9 | 46.20 | 41.6 | 37.10 | 32.8 | 28.50 |
| all | 73.00 | 72.6 | 68.70 | 63.8 | 58.90 | 54.2 | 49.60 | 44.9 | 40.40 | 35.9 | 31.50 | 27.1 | 23.00 |

| H | V | W | X | Y | Z | AA | AB | AC | AD |
|---|---|---|---|---|---|---|---|---|---|
| | AGE GROUP | | | | | | | | |
| IR_ethnicity | 55-59 | 60-64 | 65-69 | 70-74 | 75-79 | 80-84 | 85-89 | 90-94 | 95- |
| Caucasian | 19.3 | 15.80 | 12.6 | 9.80 | 7.3 | 5.30 | 3.30 | 1.30 | 1.3 |
| Caucasian | 23.0 | 19.00 | 15.3 | 12.00 | 8.9 | 6.30 | 3.70 | 1.10 | 1.1 |
| African | 19.9 | 16.70 | 13.9 | 11.20 | 9.0 | 7.00 | 5.30 | 3.60 | 3.6 |
| African | 24.5 | 20.70 | 17.2 | 13.90 | 11.2 | 8.50 | 6.20 | 3.90 | 3.9 |
| all | 19.2 | 15.70 | 12.5 | 9.80 | 7.3 | 5.40 | 3.50 | 1.60 | 1.6 |

The life expectancy LE is subtracted 273 from the age to obtain the expected remaining years of survival. The yearly risk 1YRiskₖ is determined 275 for each integer value of k=age to LE by applying Equations 7 and 8.

$$\begin{matrix}{1 Y R i s {k}_{k} = \frac{1}{1 + {e}^{- c}} \vert}_{k = a g e}^{L E , i n c = 1} \\ w h e r e & ( Eq . 7 ) \\ c = ( P O d d s + {\sum }_{j = 1}^{m} ( {b}_{j , k} ( {X}_{j} - {P}_{k} ) ) ) , a n d & ( E q . 8 )\end{matrix}$$

and where k is the index representing the age which is being increased in one-year increments up to the point of life expectancy LE. The 1-year event-free survival 1YSurvival rates for each year 1 are determined 277 according to Equation 9.

1YSurvivalₗ=(1−1YRiskₗ)|_{l=age}^{x} (Eq. 9)

The survival rate for x years is then determined 279 using Equation 10. This represents the likelihood of not having a hip fracture over the next x years.

xYSurvival=(1YSurvival₁)·(1YSurvival₂) . . . (1YSurvivalₓ) (Eq. 10)

The equivalent xYRisk is calculated 281 from Equation 11.

xYRisk=1(xYSurvival) (Eq. 11)

These operations may be performed for various values of x, such as x=5, 10, and LE. Finally, the resulting risk assessments are displayed 283.

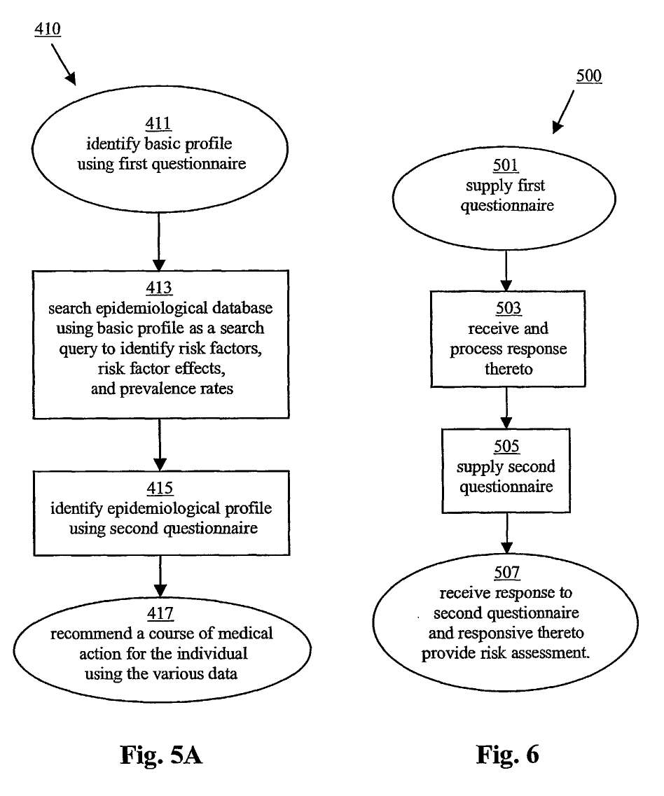

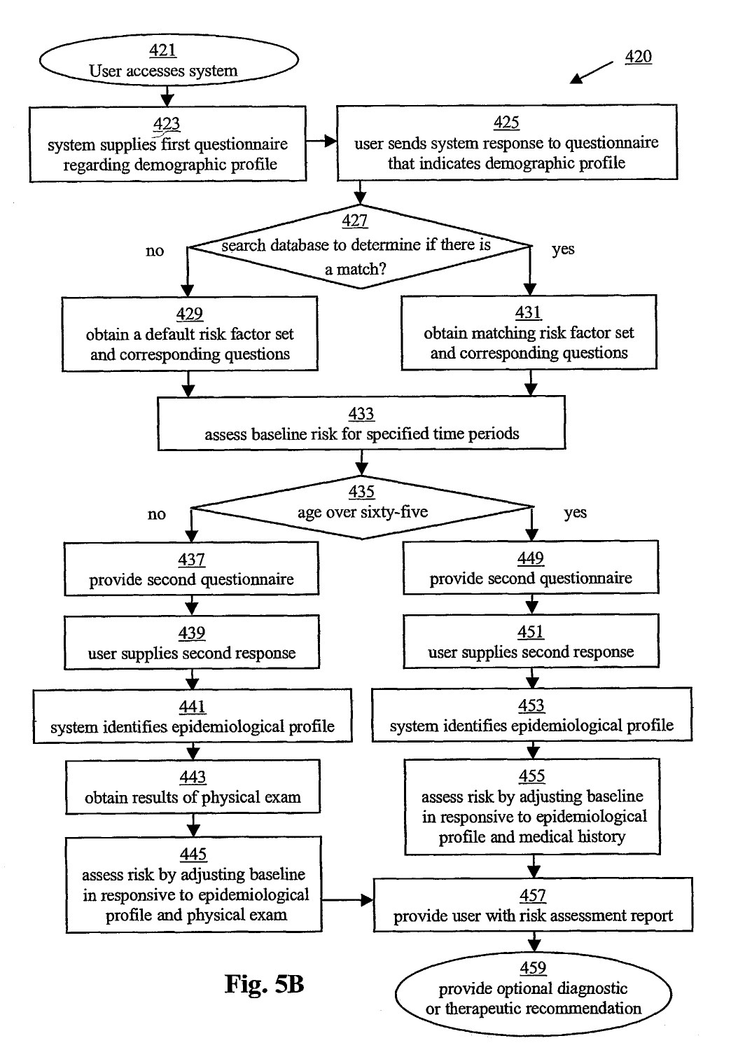

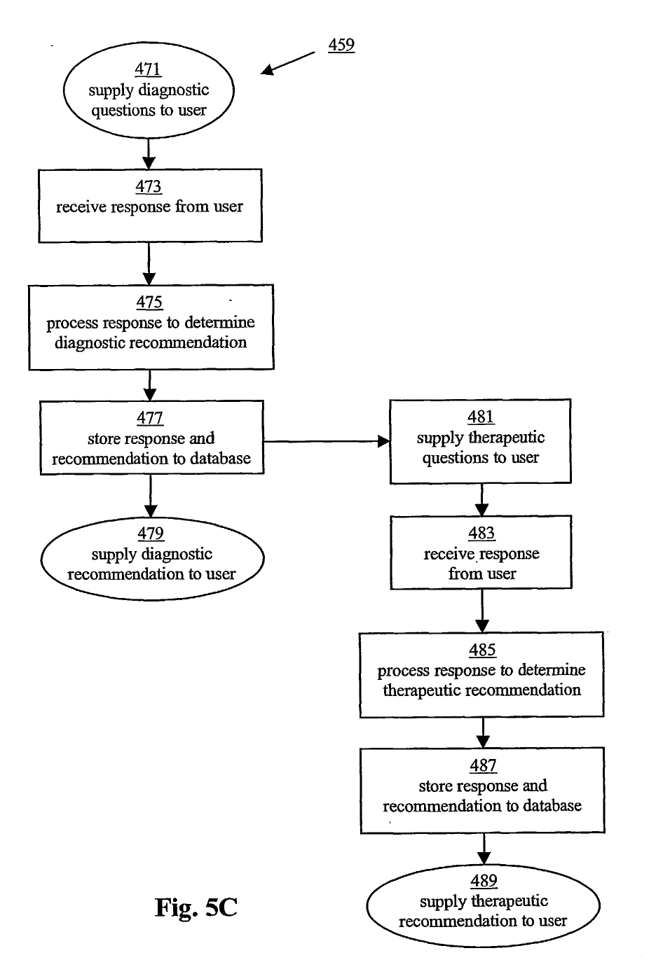

Referring now to FIG. 5A, there is shown a flowchart illustrating a first embodiment of a method 410 of recommending a course of medical action for an individual. The method 410 uses a database of epidemiological data that has been extracted from peer-reviewed scientific literature. The method 410 also uses a database of diagnostic and therapeutic information that has been extracted from peer reviewed scientific literature, library holdings, and professional medical databases such as MEDLINE.

A basic profile of the individual is identified 411 using a questionnaire. The basic profile may include demographic such as the age, gender, ethnicity, and/or geographic region of residence of the individual. Alternatively, the basic profile may include values of non-demographic parameters such as whether the individual smokes. Both demographic and non-demographic parameters may be included in the basic profile.

The database of epidemiological data is searched 413 using the basic profile as a search query. The search identifies a set of risk factors and corresponding risk factor effects that pertain to people that have substantially the same basic profile as the individual.

A second profile of the individual is identified 415 using a second questionnaire. The second profile provides data from which the values for the identified risk factors for the individual can be determined. The profiles are used to determine a course of medical action for the individual. This may include a recommended diagnostic test or course therapy. The second profile may be updated in response to such tests and/or treatments. The profiles may also be used to determine the risk that the individual will experience one or more specified outcomes over one or more periods of time.

Referring now to FIG. 5B, there is shown a flowchart illustrating another embodiment of method 410. This embodiment uses the risk assessment system 200 discussed above. The questionnaires presume that there will be results of a recent physical examination for all individuals, and that results of the individual's medical history, lifestyle, family history, and present medical state, are available for those individuals over sixty-five.

In this embodiment, a user uses a computer terminal to access 421 a server that hosts the risk assessment system 200. The user may be directed through a user identification subroutine that verifies that the user has authority to log into the system 200. A conventional password may be utilized to identify that the user has such authority and/or to indicate whether the user's level of medical expertise.

The system 200 accesses the questionnaire database 224 to obtain a first questionnaire. This questionnaire asks the user to provide the data for a basic profile of the individual. This questionnaire is like the demographic questionnaire except that: questions regarding some demographic parameters may be omitted; and some additional parameters, such as whether or not the individual smokes, may be added. The questionnaire-is supplied 423 to the user.

The user sends 425 a response to the system 200. If the response is not complete, the risk assessment system 200 sends a message to the user that identifies which data is missing from the basic profile, and provides a data entry screen for supplying the missing data. The risk assessment system 200 processes the user's response to identify the basic profile. The basic profile serves as a search query for searching the databases of the system 200. Some or all of the data for the basic profile may alternatively be obtained from the patient characteristic database 218.

The system 200 searches 427 the risk factor database 212 using the demographic profile as a search query. This determines whether there is epidemiological data that corresponds to the demographic profile. If there is not, a default set of risk data and corresponding epidemiological questions are obtained 429 from databases 212, 214, 224. The basic profile is matched to a study group as nearly as is possible. For example, an individual may be treated as though he is seventy when he is actually sixty-five. Risk calculations may then be adjusted, for example, according to the average change in rates that a five-year adjustment typically yields. The user may be told what adjustments have been made. If there is relevant data, the corresponding risk factors and risk factor effects, prevalence rates, and corresponding questions are identified 427, and obtained 431 from databases 212, 214, 224.

The system 200 assesses 433 the baseline risk that the individual will experience each of the outcomes identified in the best matching study. The baseline risk is assessed for one or more time periods. A one-year baseline risk may be assessed, for example, using Equations 2-3.

The system 200 reviews the demographic profile and determines 435 whether the individual is over age sixty-five. If the individual is not, the system 200 obtains a second questionnaire from the questionnaire database 224. This questionnaire is similar to the epidemiological questionnaire for the demographic group, except that any questions pertaining only to risk factors whose values are specified in the basic profile may be omitted.

The user sends 439 a response to the second questionnaire. In a first operating mode, if data is missing, the system 200 sends a message to the user that identifies which data is missing. In a second operating mode, if data is missing, the system 200 asks the user to verify that the data was not omitted by accident. If not, the system 200 uses average values for the persons having the same basic profile as the individual. The system 200 identifies 441 an epidemiological profile for the individual from the user-supplied data, any used average values, and the retrieved epidemiological data.

The system 200 obtains 443 the results of the individual's most recent physical exam. These results are provided by the treating physician. They may be stored in the patient characteristic database 218. The system 200 determines 445 the risk that the individual will experience each of the identified over the various time periods. This is achieved by using Equations 4 through 11 to adjust the baseline risk (or risks) assessed in act 433 using the epidemiological profile and physical examination results.

If the individual is over the age of sixty-five, the system 200 obtains another questionnaire from the questionnaire database 224. This questionnaire is similar to the epidemiological questionnaire for the demographic group, except that any questions pertaining only to risk factors whose values are specified in the basic profile may be omitted. There are also questions pertaining to past medical experiences and disease events known to substantially affect the risks associated with osteoporosis. The system 200 provides 437 the user with this questionnaire. The user supplies 451 a response that should include all the data requested. It is valuable to provide as much detail as possible for this age group because past medical experiences and disease events are known to substantially affect the risks of osteoporosis. The user may also supply detail about the individual's medical history, family history, present illnesses (if any), and past and current lifestyle.

The risk assessment system 200 identifies 453 an epidemiological profile for the individual from the user-supplied data, any used average values, and the retrieved epidemiological data. The system 200 assesses 455 the risk that the individual will experience each of the identified outcomes over the next one, five, and ten years, and over the remainder of life. The is achieved using Equations 4 through 11 to adjust the baseline risk assessed in act 433.

Regardless of the individual's age, if a particular study does not address physical examination results, an estimate of their effect is determined to supplement the assessment based on standard medical principles. A risk assessment report is provided 457 to the user that includes the assessed results.

In act 459, the risk assessment system optionally provides diagnostic or therapeutic recommendations. Diagnostic recommendations may include an indication regarding whether a particular diagnostic test should be performed. One option is to base the recommendation on the assessed levels of risk. Another option is to base the recommendation directly on the demographic and epidemiological profiles without necessarily performing the risk assessment act. For instance, past history of bone fractures in the individual's family history may indicate that a Bone Mineral Density test should be performed regardless of the value of any assessed level of risk. Therapeutic recommendations may alternatively be made in similar manner. Additional discussion on these features of the invention is provided below.

Referring now to FIG. 5C, there is shown a flowchart illustrating an embodiment the optional act 459 that provides the diagnostic and/or therapeutic recommendations. The system 200 asks the user whether a diagnostic recommendation is desired. If the user answers yes, the system 200 obtains a set of diagnostic questions from the expert recommendation database 226. The appropriate set of questions is determined using the basic and epidemiological profiles as a search query.

The system 200 may already be aware of some of the individual's medical data. If so, any corresponding questions are removed from the set. The set of diagnostic questions is supplied 471 to the user. The user reviews the questions and supplies responses that may include data about the individual's medical history and any diagnostic tests that are to be excluded from consideration. The response is received 473 by the risk assessment system 200. The risk assessment system 200 processes the response to determine 475 an appropriate diagnostic recommendation for the individual. This may be achieved according to an algorithm indicated in stored software or by looking up a matching recommendation in a stored matrix. The system 200 stores 477 the response from the user and the determined diagnostic recommendation in the patient characteristic database 218 for future reference, and supplies 479 a diagnostic report that supplies the diagnostic recommendation and any known contra-indications to its use.

In act 477, the system 200 also asks the user whether a therapeutic recommendation is desired. If the user answers yes, the system 200 reads the expert recommendation database 226 to obtain a set of therapeutic questions. The appropriate set of therapeutic questions is determined in response to the demographic and epidemiological profiles of the individual and the user's response to the diagnostic questions. As such, the system 200 may already be aware of much of the medical data that characterizes the individual. Thus, some of the therapeutic questions may already have been answered, and accordingly, may be deleted from the set. The set of therapeutic questions is supplied 481 to the user. The user reviews and questions and supplies a response which may include, for example, data about the current therapy which the individual is under, and any known contraindications to therapies, and any therapies which are to be excluded from further consideration. The response is received 483 by the system 200. The system 200 processes the response to determine 485 an appropriate therapeutic recommendation for the individual. This may be achieved according to an algorithm indicated in stored software or by looking up a matching recommendation in a stored matrix. The risk assessment system 200 stores 487 the response from the user and the determined therapeutic recommendation in the patient characteristic database 218 for future reference, and supplies 489 a therapeutic report that includes the therapeutic recommendation and any known contraindications to its use.

Referring now also to FIG. 6, there is shown a flow chart that illustrates an embodiment of a method of promoting business 500 on a computer network. For method 500, the communications link 240 is carried in part by the Internet. A user of terminal 230 accesses the data processing engine 220 via the Internet. The data processing engine 220 supplies 501 a first online questionnaire via the Internet to terminal 230. The first online questionnaire requests data to identify demographic parameters of an individual to be assessed. The user supplies his or her demographic parameters, such as the individual's age, gender, ethnicity, and location. The data processing engine 220 receives and processes 503 the data to associate it with one of the populations identified in the peer reviewed scientific literature. The data processing engine 220 supplies 505 a second online questionnaire via the communication link 240 to terminal 230. The second online questionnaire asks for values of the risk factors for the individual. The user at terminal 230 supplies this data via the Internet to the data processing engine 220. The data processing engine 220 receives and processes the data to provide 507 an assessment that the individual will have the corresponding outcome within a specified period of time. The data received from the user may be stored in the patient characteristic database 218.

The method 500 may provide risk assessment for the general public or members of the medical community. This is expected to increase traffic on a web site hosting the risk assessment system 200. The identity of a business entity that pays for the site is preferably included on the site to increase their name recognition. Moreover, use of the data processing engine is expected to identify medical needs sooner than conventional medical practice, thereby increasing the market demand for medical goods and services related to the various outcomes for which risk assessment is provided. For example, the risk assessment method 400 may be used to screen users for potential benefit of expensive medical tests related to the identified outcomes, thereby increasing demand for such tests.

The risk assessment systems 100, 200 may be tested for quality. For example, general tests may be performed to check for overall consistent quality of data in the databases and their appropriateness for an intended range of users as follows. The data and extracted questions and answers are checked for spelling, punctuation and grammatical errors. The clarity and order of text is reviewed. The format of data is also tested. Paragraphs are reviewed to determine whether their layout and font is proper and consistent. The databases are also tested to determine whether they perform properly to within established tolerances. Testing may be conducted on a random or substantially random sample of the stored data using conventional statistical techniques.

Verification tests may be performed to assure that the data in the databases is substantially free of data errors, for example, as follows. All data that is newly added to the databases is subjected to verification tests. For data that pertains to formulations and calculations that are standard and well established in the medical profession, such as those for lifetime fracture risk, verification and validation of data is performed on a one-time basis. Replacement or new data is likewise tested for quality. Study errors are corrected where the proper correction is known. Where such errors are known to exist but their corrections are unknown, the study may be removed from the database. Tolerances for the various tests are established according to the client's needs. Testing preferably meets all applicable and International Organization for Standardization (ISO) and United States Food and Drug Administration (USFDA) requirements and suggestions.

The risk assessment system 200 may further be tested to determine whether it performs properly, for example, as follows. A general test is performed to check for overall consistent provision of the questionnaires. Text in the questionnaires is checked for spelling, punctuation, and grammatical errors. The clarity and order of the data entry screens is reviewed. A verification test is performed to assure that the system 200 is stable. The results of risk assessment computations and recommendations are also verified. This may be implemented using a nested set of loops to run through various possible combinations of user inputs. The results are analyzed for accuracy by a trained staff of statisticians and physicians A validation test is performed on the system 200 to assure that it operates within client specified tolerances. This may be implemented, for example, by generating data that represents a plurality of individuals. These individuals preferably represent the type of target population for the risk assessment system 200. Data for these individuals is supplied via the questionnaires, and the result is checked against the client's specification. This testing may be conducted on a random or substantially random sample of the stored data using conventional statistical techniques. Compliance with International Organization for Standardization (ISO) and United States Food and Drug Administration (USFDA) requirements preferably is also verified.

1. A method generating a database, the method comprising:
searching peer-reviewed scientific literature to identify a class of studies that include data regarding risk factors for experiencing an outcome specific to a disease entity for members of a demographic group;
extracting said data from the studies in a form that accounts for any interdependencies among the risk factors; and
storing the extracted data indexed by the demographic group.

2. The method of claim 1, wherein the acts of searching, extracting, and storing are repeated for at least one additional demographic group.

3. The method of claim 1, wherein the peer-reviewed scientific literature is searched in accordance with Cochrane criteria.

4. The method of claim 1, further comprising the act of removing each study having a reliability beneath a specified threshold from the literature.

5. The method of claim 1, wherein the extracted data comprises a plurality of risk factors that affect the risk for members of the demographic group.

6. The method of claim 1, wherein the extracted data comprises a plurality of risk factor effects that correspond to a plurality of risk factors that affect the risk for members of the demographic group.

7. The method of claim 1, wherein the extracted data comprises a plurality prevalence rates that correspond to a plurality of risk factors that affect the risk for members of the demographic group.

8. The method of claim 1, wherein the extracted data comprises a plurality of risk factor effects, each risk factor effect having a corresponding risk factor that affects the risk for members of the demographic group.

9. The method of claim 1, wherein the extracted data indicates how a risk factor affects the risk for members of the demographic group.

10. The method of claim 1, wherein the extracted data indicates how a plurality of risk factors affect the risk for members of the demographic group.

11. The method of claim 1, wherein the extracted data comprises a risk factor and a prevalence rate, and wherein the risk for a member of the demographic group is a function of a difference between the prevalence rate and an extent that the member exhibits the risk factor.

12. The method of claim 1, wherein:
the extracted data comprises a risk factor, a risk factor effect, and a prevalence rate, and
the risk for a member of the demographic group is a function the risk factor effect, the prevalence rate, and an extent that the member exhibits the risk factor.

13. The method of claim 12, wherein the risk for said member of the demographic group is a function of an average incidence of the outcome among members of the demographic group.

14. The method of claim 8, wherein at least one risk factor effect is adjusted in value to reflect interdependency of a corresponding risk factor with at least one other risk factor.

15. The method of claim 1, wherein the extracted data comprises a plurality of risk factors for experiencing the outcome, and for each risk factor, risk data that indicates an effect on the risk for an arbitrary member of the demographic group, the effect being a function of an extent by which said member exhibits the risk factor.

16. The method of claim 1, wherein at least one study in the class analyzes only one risk factor for the demographic group.

17. A method of assessing a risk that an individual will experience an outcome specific to a disease entity within a specified period of time, said method using a database of epidemiological data extracted from peer-reviewed scientific literature, the method comprising:
identifying a demographic profile that specifies a demographic group to which the individual belongs;
determining a baseline risk that the individual will experience the outcome over the specified period of time in response to the demographic profile;
searching the database to identify a best matching study in said literature for the demographic group;
retrieving from the database any epidemiological data for said demographic group that was extracted from the best matching study;
identifying an epidemiological profile that provides values for any individual specific variables included in the identified epidemiological data; and
adjusting the baseline risk in response to the values and epidemiological data to generate the assessed risk.

18. A method of assessing a risk that an individual will experience an outcome specific to a disease entity within a specified period of time, the method comprising:
identifying a demographic profile that specifies a demographic group to which the individual belongs;
determining a baseline risk that the individual will experience an outcome specific to a disease entity over a specified period of time in response to the demographic profile;
searching a database of risk factors extracted from peer-reviewed scientific literature to identify a plurality of risk factors for that demographic group and outcome, the risk factors extracted from a best matching study in said literature for that demographic group;
identifying an epidemiological profile that provides values for the identified risk factors for the individual; and
adjusting the baseline risk in response to the values to generate the assessed risk.

19. The method of claim 18, wherein the act of searching also identifies a plurality of risk factor effects for that demographic group and outcome in the best matching study, and wherein the act of adjusting is responsive to the identified risk factor effects.

20. The method of claim 18, wherein the baseline risk is adjusted in response to an extent that a risk factor is exhibited by the individual.

21. The method of claim 18, wherein the baseline risk is adjusted in response to an extent that a risk factor is exhibited by the individual relative to an extent that the risk factor is generally exhibited by members of the demographic group.

22. The method of claim 18, wherein the individual is screened for anticipated benefit of a specified diagnostic test in response to the assessed level of risk.

23. The method of claim 18, further comprising:
specifying a threshold level of risk of experiencing the specified outcome; and
comparing the assessed risk to the threshold level of risk to determine whether to perform the specified diagnostic test on the individual.

24. The method of claim 23, wherein the threshold level of risk is specified in response to an anticipated net benefit of performing a specified diagnostic test on individuals whose risk of experiencing the specified condition is at least equal to the threshold level of risk.

25. The method of claim 18, wherein the assessed level of risk is used to screen the individual for likely benefit of a specified therapy.

26. The method of claim 25, further comprising:
specifying a threshold level of risk of experiencing the specified outcome; and
comparing the assessed risk to a threshold level of risk to determine whether to perform the specified therapy on the individual.

27. The method of claim 26, wherein the threshold level of risk is specified in response to an anticipated net benefit of performing a specified therapy on individuals whose risk of experiencing the specified condition is at least equal to the threshold level of risk.

28. The method of claim 18, wherein a diagnostic test is recommended to the individual in response to the assessed risk.

29. The method of claim 18, wherein a therapy is recommended to the individual in response to the level of assessed risk.

30. The method of claim 18, further comprising:
searching a database of therapeutic data in response to the demographic profile to identify a therapy for the individual;
receiving a medical history of the individual; and
recommending a course of therapy for the individual in response to the identified therapy and medical history.

31. The method of claim 30, wherein the medical history is analyzed to determine whether there are contra-indications to the identified therapy, and the identified therapy is recommended for the individual in response to a lack of such contra-indications.

32. The method of claim 30, wherein the medical history is analyzed to determine whether there are contra-indications to the identified therapy, at least one contra-indication is determined, and the identified therapy is recommended for the individual in response to the level of risk specific to the determined contra-indication.

33. The method of claim 18, wherein a first questionnaire is supplied that asks for the individual's demographic group, and wherein a response by the user to the first questionnaire serves as the demographic profile.

34. The method of claim 33, wherein a second questionnaire that includes a plurality of questions for values of the identified plurality of risk factors for the individual is supplied, and wherein a response by the user to the second questionnaire serves as the epidemiological profile.

35. The method of claim 34, wherein the content of the second questionnaire is responsive to the level of medical expertise of the user.

36. The method of claim 34, wherein the content of the second questionnaire is responsive to the level of scientific expertise of the user.

37. The method of claim 18, wherein an average value characterizing individuals having the same demographic profile the individual with respect to a given identified risk factor is used in response to failure of the user to characterize the individual with respect to the given identified risk factor.

38. The method of claim 18, wherein the specified period of time is an interval of time ending at a specified time in the future.

39. The method of claim 18, wherein a memory unit stores instructions for performing the method and wherein a processing unit coupled with the memory unit receives the instructions from the memory unit and in response thereto performs at least one step of the method.

40. The method of claim 18, wherein the method is implemented by a general-purpose computer.

41. The method of claim 18, wherein the method is implemented by a personal digital assistant.

42. The method of claim 18, wherein at least one step of the method is implemented by a processing unit in response to instructions embedded in the processing unit.

43. A method of recommending a course of medical action for an individual, said method using a database of risk data that includes data regarding risk factors and corresponding risk factor effects, said risk data having been extracted from peer-reviewed scientific literature, the method comprising:
identifying a first set of data that characterizes an individual in relation to a plurality of demographic parameters;
searching a database of epidemiological data extracted from peer-reviewed scientific literature in response to the first set of data to identify a plurality of risk factors and corresponding risk factor effects that are adjusted for interdependency among the risk factors;
identifying a second set of data that characterizes the individual in relation to the plurality of risk factors; and
in response to the sets of data, recommending a course of medical action for the individual.

44. A method of promoting business on a computer network, said method comprising supplying a first online questionnaire to a user regarding demographic characteristics of an individual, receiving a response to the first questionnaire and in response thereto searching epidemiological data extracted from peer-reviewed scientific literature to identify a plurality of risk factors that affect the likelihood of the individual experiencing a specified outcome, supplying a second online questionnaire to the user regarding the epidemiological characteristics of the individual relative to the identified plurality of risk factors, receiving a response from the user to the second questionnaire, and assessing risk for the individual to experience a specified outcome within a disease entity within a specified period of time in response to both responses from the user.

45. The method of claim 44, wherein the online questionnaire is supplied over a computer network.

46. The method of claim 44, wherein the online questionnaire is supplied over the Internet.

47. The method of claim 44, wherein the online questionnaire is supplied over an Intranet.

48. The method of claim 44, wherein the online questionnaire is supplied over an Extranet.

49. A computer programmed to receive a demographic profile of an individual; responsive to the profile, to supply a questionnaire regarding a plurality of risk factors specific to a specified disease entity; and responsive to a response to the questionnaire, to supply an assessment of the risk of the individual experiencing at least one outcome specific to the specified disease entity over at least one specified period of time.

50. The computer of claim 49, wherein the assessment accounts for interdependency among the plurality of risk factors.

51. A medium readable by a machine, the medium carrying a program of instructions executable by the machine for performing a method of assessing risk for an individual to experience a specified outcome (associated with a disease entity) within a specified period of time, said method using a database of risk factors extracted from peer-reviewed scientific literature, the method comprising:
identifying a demographic profile that specifies a demographic group to which the individual belongs;
determining a baseline risk that the individual will experience an outcome specific to a disease entity over a specified period of time in response to the demographic profile;
searching a database of risk factors extracted from peer-reviewed scientific literature to identify a plurality of risk factors for that demographic group and outcome, the risk factors extracted from a best matching study in said literature for that demographic group;
identifying an epidemiological profile that provides values for the identified risk factors for the individual; and
adjusting the baseline risk in response to the values to generate the assessed risk.

52. A medium readable by a machine, the medium carrying a program of instructions executable by the machine for performing a method of assessing risk of an individual experiencing a outcome within a disease entity within a specified period of time, the method comprising:
identifying a first set of data that characterizes an individual in relation to a plurality of demographic parameters;
searching a database of epidemiological data extracted from peer-reviewed scientific literature in response to the first set of data to identify a plurality of risk factors and corresponding risk factor effects that are adjusted for interdependency among the risk factors;
identifying a second set of data that characterizes the individual in relation to the plurality of risk factors; and
in response to the sets of data, recommending a course of medical action for the individual.
